(19) United States
(12) Patent Application Publication
(10) Pub. No.: US 2022/0176149 A1
(43) Pub. Date: Jun. 9, 2022

(54) METHOD OF TREATMENT OR PREVENTION OF KELOIDS AND HYPERTROPHIC SCARS USING ELECTROMAGNETIC RADIATION

(71) Applicant: Emoled S.r.l., Firenze (FI) (IT)

(72) Inventor: Lorenzo TARGETTI, Firenze (FI) (IT)

(73) Assignee: Emoled S.r.l., Firenze (FI) (IT)

(21) Appl. No.: 17/111,530

(22) Filed: Dec. 4, 2020

(51) Int. Cl. A61N 5/06

(52) U.S. Cl. CPC A61N 5/0616; A61N 2005/0658; A61N 2005/0651

(57) ABSTRACT

A method is presented to prevent formation of a scar or keloid following an insult to an area of a mammalian skin creating a lesion defining an outer surface, where the lesion reaches at least the dermis of the skin. The method includes irradiating the lesion with electromagnetic radiation having a wavelength between 410 nm and 430 nm for an exposure time long enough that the energy density received by the outer surface of the lesion is comprised between 15 J/cm² and 120 J/cm².

### FIELD OF THE INVENTION

The present invention relates to the treatment or prevention of keloids and hypertrophic scars using electromagnetic radiation at a specific wavelength and power. Further, the invention relates to the treatment of already developed keloids and hypertrophic scars.

### BACKGROUND

Keloids (or hypertrophic scars) are an overgrowth of fibrotic tissue outside the original boundaries of an injury and occur secondary to defective wound healing. Keloids vary in size, density, demarcation, and site. The exact pathogenesis of keloids is not elucidated and the lack of adequate animal models hinders keloid research.

Excessive scars form as a result of aberrations of physiologic wound healing and may develop following any insult to the deep dermis, including burn injury, lacerations, abrasions, surgery, piercings and vaccinations. By causing pruritus, pain and contractures, excessive scarring can dramatically affect a patient's quality of life, both physically and psychologically. Hypertrophic scarring has an incidence between 40% and 70% following surgery, up to 90% in case of burns. Furthermore, keloids have an incidence of 6% in Caucasian population and up to 16% in African Population. Moreover, keloids are prone to high recurrence (50%-80%) in patients suffering from them.

Keloids often have a functional, aesthetic, or psychosocial impact on patients as highlighted by quality-of-life studies.

Treatments for keloids include surgical excision, intralesional or topical corticosteroids, other intralesional therapies, compression, cryotherapy, radiation, silicon sheeting, and laser or light-based therapies. Recurrence is common, even with combination therapy. Lasers and other light-based technology have introduced new ways to manage keloids that may result in improved aesthetic and symptomatic outcomes and decreased keloid recurrence. Laser and light-based therapies for keloids can be grouped into three categories: ablative lasers, non-ablative lasers, and non-coherent light sources.

However, the scar treatment products and methods known to date may not be efficient at treating all scar types. As such, there remains a need in the art for additional scar treatment methods that will allow patients and health care practitioners to decide on the most efficient scar method treatment for a given type of scar or even before the formation of the scar.

It is thus an object of the present disclosure to propose a method for treatment or prevention of keloids and scars.

The term “preventing” or “prevention” as used herein in the context of preventing a scar or prevention of a scar, refers to eliminating, ameliorating, decreasing or reducing a scar/keloid or development of a scar/keloid. The term “treating” or “treatment” as used herein the context of treating a scar or treatment of a scar/keloid, refers to having a therapeutic effect and at least partially alleviating or abrogating or ameliorating a scar/keloid.

As used herein, the terms “reduce”, “improve”, “promote”, “facilitate” and similar comparative statements is meant to compare the condition where the patient is treated with the invention to the untreated state.

In the following, the term “scar” indicates a mark remaining on the skin after the healing of a wound, such as one caused by injury, illness, smallpox vaccination, or surgery. Beneath the skin is a fibrous connective tissue known as subcutaneous tissue, composed of cells called fibroblasts, which after injury are stimulated to grow into granulation tissue, knitting the wound together. Scar tissue is formed by dense masses of granulation tissue. The scar is called also cicatrix.

A “keloid” indicates a sharply elevated, irregularly shaped, progressively enlarging scar, due to excessive collagen formation in the corium during connective tissue repair. It is a benign tumor that usually has its origin in a scar from surgery or a burn or other injury.

Three distinct phases involved in the pathophysiology of excessive scar formation have been described: inflammation, proliferation and remodeling. In normal scar healing, during the inflammation phase, platelet degranulation will be responsible for the release and activation of an array of different potent cytokines which will serve as chemotactic agents to recruit macrophages, neutrophils, epithelial cells and fibroblasts. In normal conditions, a balance will be achieved between new tissue biosynthesis and degradation mediated by apoptosis and remodeling of the extracellular matrix. In hypertrophic scarring, a persistent inflammation, caused by an increased secretion of different factors (e.g., TGF-β1, TGF-P2, PDGF, IGF-1, IL-4 and IL-10) might lead to an excessive collagen synthesis or deficient matrix degradation and remodeling. Scars are classified into different categories, based on the nature of the injury having caused the scar, its clinical characteristics and its appearance. Flat or pale scars (known as linear scars) are the most common type of scar and result from the physiological healing process. Initially, these scars may be red or dark and raised after the wound has healed but they will eventually become paler and flatten naturally over time, resulting in a flat, pale scar. This process can take up to two years and there will always be some visible evidence of the original wound. Hypertrophic scars are more common in young people and people with darker skin.

When a normal wound heals, the body produces new collagen fibers at a rate which balances the breakdown of old collagen. Hypertrophic scars are red and thick and may be itchy or painful. They do not extend beyond the boundary of the original wound but may continue to thicken for up to 6 months. They usually improve over the next one to two years but may cause distress due to their appearance or the intensity of the itching, also restricting movement if they are located close to a joint. It is not possible to completely prevent hypertrophic scars. Similar to hypertrophic scars, keloids are the result of an imbalanced collagen production in a healing wound. Unlike hypertrophic scars, keloids grow beyond the boundary of the original wound and can continue to grow indefinitely. They may be itchy or painful and most will not improve in appearance over time. Keloid scars can result from any type of injury to the skin, including scratches, injections, insect bites and tattoos. Some parts of the body are more sensitive to the development of keloids, such as ears, chest, shoulders and back.

An “insult” is a bodily injury, irritation, or trauma.

### SUMMARY

According to an aspect, the invention relates to a method to prevent formation of a hypertrophic scar or keloid following an insult to an area of a mammalian skin creating a lesion defining an outer surface, wherein the lesion reaches at least the dermis of the skin, the method comprising: irradiating the lesion with electromagnetic radiation having a wavelength comprised between 410 nm and 430 nm for an exposure time long enough that the energy density received by the outer surface of the lesion is comprised between 15 J/cm² and 120 J/cm².

According to another aspect, the invention relates to a method of treatment of an already formed hypertrophic scar or keloid on mammalian skin to avoid its recurrence, the method comprising: surgically removing the hypertrophic scar or keloid to form an area of the skin with a lesion defining an outer surface which reaches the dermis, and irradiating the lesion with electromagnetic radiation having a wavelength comprised between 410 nm and 430 nm for an exposure time long enough that the energy density received by the outer surface of the lesion is comprised between 15 J/cm² and 120 J/cm².

The method of the invention, in either aspect, is to be performed on mammalian skin, preferably on the human skin. The mammalian skin, as known, is composed of two primary layers: the epidermis, which provides waterproofing and serves as a barrier to infection; and the dermis, located underneath the epidermis. The epidermis is composed of the outermost layers of the skin. It forms a protective barrier over the body's surface, responsible for keeping water in the body and preventing pathogens from entering, and is a stratified squamous epithelium, composed of proliferating basal and differentiated suprabasal keratinocytes. The dermis is the layer of skin beneath the epidermis that consists of connective tissue and cushions the body from stress and strain. The dermis provides tensile strength and elasticity to the skin through an extracellular matrix composed of collagen fibrils, microfibrils, and elastic fibers embedded in hyaluronan and proteoglycans. It harbors many mechanoreceptors (nerve endings) that provide the sense of pain, touch and heat through nociceptors and thermoreceptors. It also contains the hair follicles, sweat glands, sebaceous glands, apocrine glands, lymphatic vessels and blood vessels. The blood vessels in the dermis provide nourishment and waste removal from its own cells as well as for the epidermis.

The epidermis and dermis are separated by a thin layer of fibers called the basement membrane, which is made through the action of both tissues. The basement membrane controls the traffic of the cells and molecules between the dermis and epidermis but also serves, through the binding of a variety of cytokines and growth factors, as a reservoir for their controlled release during physiological remodeling or repair processes.

The hypodermis lies below the dermis. Its purpose is to attach the skin to underlying bone and muscle as well as supplying it with blood vessels and nerves. It consists of loose connective tissue and elastin. The main cell types are fibroblasts, macrophages and adipocytes (the subcutaneous tissue contains 50% of body fat). Fat serves as padding and insulation for the body.

Generally, but not necessarily, in order to have a scar or keloid, a damage or insult of the dermis has taken place. Therefore, in the following, in case of prevention of the formation of a keloid or scar, or when an already formed scar or keloid is removed, the lesion which is formed in the skin is deep enough that it reaches the dermis, more preferably the deep dermis. The lesion may be so deep that it reaches the hypodermis. In the dermis and in the hypodermis, fibroblast cells are present, as detailed below.

The present invention proposes a method to prevent or minimize the formation of hypertrophic scars and keloids following any insult to the dermis. The insult can be of any type, as long as it forms a lesion on the skin reaching the dermis layer. For example, the lesion can be a wound (such as for example a surgical wound or a wound caused by trauma), burn lesion, blister or ulcer, the result of infections, etc. The only requirement for the lesion derived by the insult is that the lesion is deep enough that the dermis is involved. The lesion may also reach the hypodermis.

The method of the invention to prevent or minimize the formation of the keloid or the hypertrophic scar is to be applied to the patient before the keloid or hypertrophic scar is formed. This means that the method of the invention has to be applied before there is a complete healing of the skin. Preferably, the method of the invention has to be applied right after the insult occurrence. The method of the invention is to be applied when, on the skin, there is still a lesion present. Applying the method of the invention on the skin's lesion minimizes or avoids the formation of a keloid or hypertrophic scar where the lesion has been formed. With the terms “minimize the formation of a keloid or hypertrophic scar”, it is intended that the reconstructed skin area formed after the application of the method of the invention, that is, the skin which forms on the lesion to close the same, has a closer resemblance to the normal patient skin according to measurable clinical observations than the skin spontaneously formed without the application of the method of the invention. The lesion origin may be due to an insult, for example an injury, surgical intervention, acid presence or flame (burns).

The above method is applicable also to already formed hypertrophic scars or keloids. The hypertrophic scar or keloid is first of all removed, for example by surgical incision or ablation, and then the method is applied to the lesion formed by removing the hypertrophic scar or keloid in order to prevent the recurrence of the removed hypertrophic scar or keloid. Thus, after the removal of the already formed hypertrophic scar or keloid, the method is substantially the same as the method used to prevent or minimize the formation of a new hypertrophic scar or keloid.

The application of the electromagnetic radiation at the specified wavelength between 410 nm and 430 nm with a radiant energy density comprised between 15 J/cm² and 120 J/cm² takes place at the lesion level. That is, the electromagnetic radiation is applied to the lesion for a given time interval, called exposure time. During the exposure time, the lesion present on the skin is irradiated at the claimed radiant energy density, that is, the radiant energy density is the radiance that the outer surface of the lesion receives integrated over the exposure time. The radiant energy density is due to the irradiation of the lesion by the electromagnetic radiation having a wavelength comprised between 410 nm and 430 nm. The outer surface of the lesion is at the dermis level (or hypodermis level), because, as said, the lesion reaches at least the dermis.

Preferably, the application of the electromagnetic density is continuous, in the sense that during the exposure time in which the method takes place, there is a “continuous” application of electromagnetic radiation. “Continuous” means that the irradiation by the given electromagnetic radiation does not stop during the exposure time, i.e. there are no interruptions in the irradiation by the selected electromagnetic radiation having a wavelength between 410 nm and 430 nm. However, the electromagnetic radiation itself may be continuous or pulsed. The pulse repetition of the electromagnetic radiation is preferably comprised between 10 KHz and 400 KHz. In case of pulsed electromagnetic radiation, the fluence values between 15 and 120 J/cm² are intended as the mean of the fluence over the period of the repetition rate (i.e. number of pulses per second). Thus, when a pulsed electromagnetic radiation is used, a continuous irradiation during the exposure time means that there are no interruptions in the irradiation besides those which are intrinsic to the pulsed electromagnetic radiation's nature.

It has been shown by the Applicants that light energy at a specific wavelength and fluence may cause functional modifications to the scar or keloid fibroblast cells, as detailed below.

A fibroblast is a type of cell that synthesizes the extracellular matrix and collagen, produces the structural framework (stroma) for animal tissues, and plays a critical role in wound healing, especially in the proliferation and remodeling phases. Fibroblasts are the most common cells of connective tissue in animals. Therefore, in the formation of hypertrophic scars or keloids, the activity of fibroblasts is involved.

This amount of radiant energy density at the specific claimed wavelength causes changes in the fibroblasts present in the dermis and/or in the hypodermis of the irradiated skin. The radiant energy density to which the lesion is subjected is conceived in such a way to trigger the targeted biological reactions and to avoid an excessive increase of the dermis superficial temperature. Preferably, the temperature of the dermis or hypodermis where the lesion is present is kept at a temperature lower than 48° C. during the whole treatment. For this reason, the lesion should not be irradiated with too high power. Furthermore, too high power may kill the cells of the skin. On the other hand, outside the claimed range, in the lower part, the energy threshold to induce the photobiomodulation (detailed below) may not be achieved.

The amount of radiant energy density irradiating the outer surface of the lesion in the exposure time is more preferably comprised between 20 J/cm² and 50 J/cm². The amount of radiant energy density irradiating the outer surface of the lesion in the exposure time is even more preferably comprised between 25 J/cm² and 40 J/cm².

The electromagnetic radiation has a wavelength comprised between 410 nm and 430 nm, more preferably comprised between 415 nm and 425 nm. Although this is technically a wavelength in the violet range, it is generally called in the field “blue light” (blue electromagnetic radiation).

Preferably, the radiant flux density received by the outer surface per unit area of the lesion is comprised between 50 mW/cm² and 200 mW/cm². Preferably, during an irradiation, the radiant flux density is kept constant.

Preferably, the irradiation of the lesion by electromagnetic radiation at a wavelength comprised between 410 nm and 430 nm is performed using a LED (Light Emitting Diode). Preferably, an array of LEDs is used. Preferably, the number of LEDs in the array is comprised between 1 and 30. The electromagnetic radiation may be emitted by a LED optical fiber. In front of the LEDs, preferably a suitable optics is located in order to collimate and to even out the intensity of the resulting beam of electromagnetic radiation produced by the LEDs.

Preferably, the LEDs outputting the electromagnetic radiations on the lesion are located at a distance comprised between 1 cm and 15 cm, more preferably between 4 cm and 15 cm, from the outer surface of the lesion itself. The distance is calculated from the outer surface of the lesion to the LED source or from the tip of the optical fiber.

Preferably, the radiant flux density, that is, the radiant flux received by the outer surface of the lesion per unit area (W/m²) remains constant during the whole irradiation. Preferably, therefore, the irradiation of the lesion just takes longer (for example, a longer exposure time is selected) in case a higher radiant energy density is desired. Further, the radiant energy density, or fluence (which is the optical energy delivered per unit area), is delivered by an energy source which is so set that at the level of the outer surface of the lesion a substantially uniform “flow” of energy is present. If the lesion cannot be irradiated as a whole at the same time, for example because the area of the outer surface of the lesion is too large, several irradiations having identical characteristics are performed, so that the whole lesion's outer surface is irradiated in the same way (that is, with the same electromagnetic radiation having the same wavelength, with the same power and exposure time).

Further, preferably the irradiation of the lesion by the electromagnetic radiation lasts between 30 seconds and 600 seconds. The irradiation time, or “exposure time” is the time in which the irradiation takes place. More preferably, the irradiation lasts for a time interval comprised between 60 seconds and 360 seconds, even more preferably between 120 seconds and 300 seconds, even more preferably between 180 seconds and 240 seconds. During this time interval, the irradiation of the outer surface of the lesion by the blue electromagnetic radiation is continuous, that is, for a time interval between 30 s and 600 s the lesion is irradiated without interruptions. The term “continuous” also includes pulsed irradiation, as long as the “interruption” in the emission of electromagnetic waves is shorter than 10% of the repetition rate of the pulsed light. Thus, the exposure time above mentioned, i.e. between 30 s and 600 s, includes the “interruptions” due to the pulsed nature of the electromagnetic radiation. For example, due to the fact that the pulsed light has a pulse comprised between 10 kHz and 400 KHz, the interruption in order not to be counted as such should not be longer than 10⁻³ seconds. In other words, the electromagnetic radiation in the present invention has to be pulsed “fast enough” to have a repetition rate above 10 kHz. In this case, the pulsed irradiation is considered as equivalent to a continuous irradiation. During the exposure time, the surface of the lesion, either caused by the removal of the keloid/scar or by an insult, is irradiated substantially uniformly, that is, all points in the outer surface of the lesion at the same distance from the LED receive the same radiant energy density. The specific exposure time used to irradiate the lesion may depend on one or more of: color of the skin (according to Fitzpatrick scale) in which the lesion is present, patient risk in developing keloid (for example, if the patient has a history of recurrent keloid), aetiology of the lesion (for example whether it is surgical, spontaneous, burns, injury), inflammation level of the area where the lesion is present, comorbidities of the patient (for example whether the patient is diabetic, has vascular insufficiency, of he/she has any inflammation type of disease), depth of the lesion.

The number of treatments, that is, the number of irradiations as described above using a electromagnetic radiation at a wavelength of between 410 nm and 430 nm and achieving a radiant energy density (or fluence) at the outer surface of the lesion equal to between 15 J/cm² and 120 J/cm², depends on the lesion characteristics, origin and aetiology as well as lesion status according to the T.I.M.E. scale. Preferably, the number of irradiations performed is comprised between 3 and 10, more preferably between 4 and 8, even more preferably between 4 and 6. In each treatment (i.e. in each irradiation), the lesion is irradiated by an electromagnetic radiation at a wavelength comprised between 410 nm and 430 nm with a power and for an exposure time such that an energy density (or fluence) at the outer surface of the lesion equal to between 15 J/cm² and 120 J/cm² is obtained. Preferably, each of the irradiations of the lesion lasts between 30 seconds and 600 seconds. More preferably, each of the irradiations lasts for a time interval comprised between 60 seconds and 360 seconds, even more preferably between 120 seconds and 300 seconds, even more preferably between 180 seconds and 240 seconds. Preferably, in order to have the best results, more than a single treatment (=irradiation) of the outer surface of the lesion takes place.

Furthermore, besides the total number of treatments, the Applicants have found that also a certain frequency of treatments is preferred. Preferably, between one irradiation and the next subsequent irradiation, both irradiations being performed according to the invention (wavelength between 410 and 430 nm, an energy density at the surface of the lesion equal to between 15 J/cm² and 120 J/cm²), a “recovery time interval” elapses. Preferably, this “recover time interval” is of at least 48 hours, that is, the temporal distance between two subsequent irradiations is of at least 48 hours. Preferably, the temporal distance between two subsequent irradiations is comprised between 48 hours and 96 hours, more preferably between 48 hours and 72 hours, even more preferably between 48 hours and 52 hours. Between two subsequent irradiations, no irradiation takes place. With “no irradiation”, it is meant that no irradiation with the blue light at the claimed wavelength and energy density takes place. Of course, the lesion can be exposed to standard light present in the environment. Standard environment light also comprises a blue component, however the power of such component is much lower than the power used in the treatment of the invention. The irradiation of the lesion achieves the “peak” of effectiveness after about 48 hours from the irradiation itself, as shown by the experimental data below. The total number of irradiations, and their frequency, depend on the lesion characteristics, origin and aetiology, on the patient's comorbidities and overall health status.

The irradiation is preferably performed using a portable device brought close to the lesion. The optics of the device is such that the irradiation on the area of interest is substantially uniform. Thus, the intensity of the electromagnetic beam in a cross section of the beam emitted by the device is substantially uniform.

The irradiation of the lesion is performed preferably using a simple device, for example using one or more LEDs which emit at the desired radiation and having the necessary power. The operator therefore irradiates the lesion for the exposure time (for example, till the desired energy density is achieved) simply holding the apparatus including the LEDs in his/her hand.

The Applicants have performed studies to understand the effect that the blue light, at the claimed radiant energy intensity, has on the fibroblasts activity, because fibroblast and their collagen production are one of the causes of scars and keloids.

In order to determine the effect that the blue light at a given radiant energy intensity has on the fibroblasts, the Applicant has performed the following studies. A first study has been performed on fibroblasts in vitro to study the effect of the blue light on them without any other structure. A specific behavior of the fibroblasts has been observed. Then, the effect of the same blue light on fibroblasts in synthetic skin has been studied in order to understand the proper radiant energy density to be used on mammalian skin to obtain the desired effect already found in the in vitro tests. Indeed, when the electromagnetic radiation enters into the skin, its intensity is reduced and therefore the proper energy density which reaches the outer surface of the lesion has to be adapted so that the desired radiant energy density is received by the fibroblasts inside the dermis or hypodermis.

The most direct means of measuring cell proliferation, a determination of the number of actively dividing cells, is to count the number of cells present. Cell viability, defined as the number of metabolically active cells in a sample, determines the number of cells (regardless of phase around the cell cycle) that are living or dead, based on a total cell sample.

A classic approach to assessing metabolic activity involves the use of tetrazolium salts that are cleaved by metabolically active cells to form colored, water-insoluble (MTT) or water-soluble (XTT, WST-1, and WST-8) formazan salts that can be measured by absorbance.

In the present invention, in order to study the metabolism and the proliferation, WST-8 and SRB assays (Sigma-Aldrich, Italy) were used to measure fibroblasts metabolism and proliferation, respectively. Further, the viability of the fibroblasts has been monitored as well, using a Trypan Blue method solution.

From the data collected by the Applicants, it is clear that the fibroblasts in vitro, if irradiated with blue light with a radiant energy density comprised between 10 J/cm² and 50 J/cm² decreases both their metabolism and proliferation, without affecting cells viability.

A higher radiant energy density (obtained for example with a higher intensity or longer exposure time) would cause cells death and therefore would damage the patient skin. Further, a lower radiant energy density (obtained for example with a lower intensity or shorter exposure time) would not give the above identified benefits, having little or no effect on the fibroblasts.

Using studies with synthetic skin and human skin, it has been shown that the above-mentioned effect seen in vitro is reproduced in mammalian skin using a blue light for an amount of time and a power set in such a way that a radiant energy density (or fluence) irradiated through the outer surface of the lesion equal to between 15 J/cm² and 120 J/cm² is obtained. The exact amount of energy density required to treat the lesion depends on the depth and type of lesion and on the characteristic of the skin of the patient.

Therefore, the claimed process using electromagnetic radiation modifies the behavior of fibroblasts in the dermis or hypodermis of the skin. These modifications, without being bound by theory, might be the reason of the reduction or prevention of scars or keloids and the avoidance of the regrowth of a scar/keloid when it is removed.

### BRIEF DESCRIPTION OF THE DRAWINGS

The invention will be in the following disclosed with non-limiting reference to the appended drawings where:

FIGS. 1 and 2 are two perspective views of an apparatus to be used in the present invention;

FIG. 2a is a perspective view of a different embodiment of an apparatus according to the invention;

FIGS. 3 and 4 show the application of the apparatus of FIGS. 1 and 2 on a skin lesion according to the method of the invention;

FIGS. 5 and 6 are photographs of similar scar burns treated (FIG. 5) and not treated (FIG. 6) with the method of the invention;

FIGS. 7A-D are graphs of the results of the irradiation according to the invention on cell metabolism in cultured fibroblasts isolated from keloid tissue and perilesional tissue. FIGS. 7A-B are the metabolism at 24 and 48 hours after the treatment in keloid fibroblasts, respectively. FIGS. 7C-D: metabolism at 24 and 48 hours after the treatment in perilesional keloid fibroblasts, respectively. Data are expressed as mean±SEM. Each measure is repeated in triplicate at 24 hours and in duplicate at 48 hours after treatment. Statistical analysis: *p<0.05; **p<0.01; ***p<0.001; ****p<0.0001 vs control (not irradiated cells), one-way ANOVA followed by Dunnett's multiple comparison test;

FIGS. 8A-D are graphs of the results of the irradiation according to the invention on cell proliferation in cultured fibroblasts isolated from perilesional tissue and keloid tissue. FIGS. 8A-B: proliferation observed 24 and 48 hours after the treatment in keloid fibroblasts, respectively. FIGS. 8C-D: proliferation observed 24 and 48 hours after the treatment in perilesional keloid fibroblasts, respectively. Data are expressed as mean±SEM. Each measure is repeated in triplicate at 24 hours and in duplicate at 48 hours. Statistical analysis: *p<0.05; ***p<0.001; ****p<0.0001 vs control (not irradiated cells), one-way ANOVA followed by Dunnett's multiple comparison test;

FIGS. 9A-D are photomicrographs of cultured human healthy fibroblasts (10× magnification) stained with Trypan blue, in different experimental groups. FIGS. 9A-B: untreated cells; FIGS. 9C-D: treated with a 41.2 J/cm2 blue light dose;

FIG. 10 are graphs of the results of the irradiation according to the invention on cell metabolism at 24 hours after the treatment in cultured fibroblasts isolated from healthy skin donor. Data are expressed as mean±SEM with constant power but with different exposure time;

FIG. 11 are graph of the same experiment data of FIG. 10 where the exposure time has been replaced by the fluence; and

FIGS. 12A-D are graphs of the results of the irradiation according to the invention on cell proliferation in cultured healthy fibroblast cells. FIGS. 12A-B: metabolism 24 and 48 hours after the treatment in fibroblasts cells, respectively. FIGS. 12C-D: proliferation 24 and 48 hours after the treatment in fibroblasts. Data are expressed as mean±SEM. Each measure is repeated in triplicate. Statistical analysis: *p<0.05; **p<0.01; ***p<0.001; ****p<0.0001 vs control (not irradiated cells), one-way ANOVA followed by Dunnett's multiple comparison test.

### DETAILED DESCRIPTION OF THE PREFERRED EMBODIMENTS

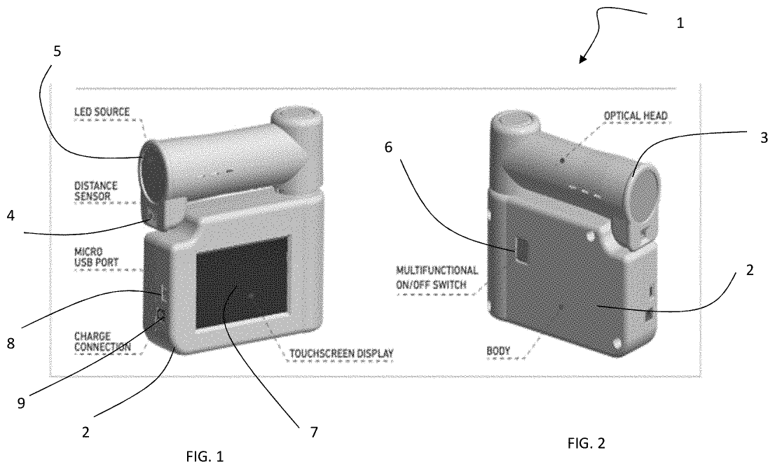

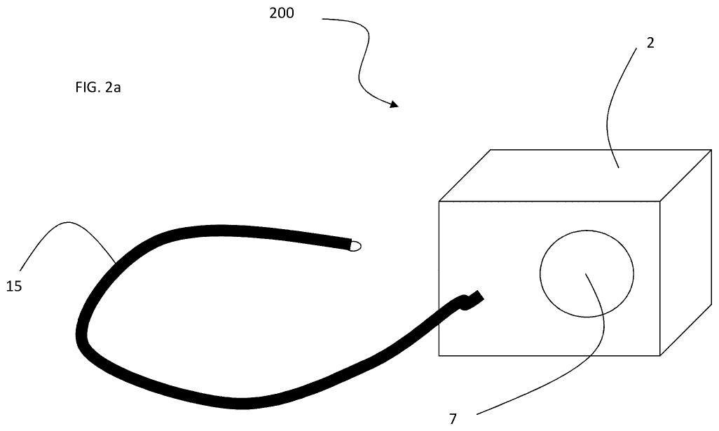

In the FIGS. 1 and 2, an apparatus 1 for the irradiation of a lesion on a patient skin is shown. The apparatus 1 includes a body 2 and a head 3. The head 3 is rotatably attached to the rest of the body 2 so that it can be oriented in several directions.

FIG. 2a shows a variant of the apparatus 200, used as apparatus 1. The apparatus 200 has a body 2 and a LED fiber optic 15.

The head 3 includes a plurality of LEDs (not visible in the drawings), specifically 6 LEDs, adapted to emit a beam of electromagnetic radiations having a wavelength comprised between 410 nm and 430 nm, as well as a suitable optics (not shown in the drawings) to collimate the beam of electromagnetic radiation and render it uniform in intensity over a cross section of the beam. The beam generated by the LEDs can exit the optics via a suitable opening 5 formed in the head 3. The wavelength of electromagnetic radiation emitted by the LEDs is preferably tunable. The maximum area that the LEDs can irradiate at the same time is equal to 20 cm².

In the apparatus 200, the electromagnetic radiation exits from the tip of fiber optic 200.

The maximum irradiance emitted by the apparatus 1 or 200, in particular by the LEDs present in the device, is equal to 120 mW/cm², which is also tunable. Therefore, the irradiance can be selected before the treatment of the lesion.

Further, preferably at the head 3, the apparatus 1 includes an optoelectronic distance sensor 4 adapted to determine the distance between the head 3 and a portion of a body of a patient as described below. Apparatus 200 may include a distance sensor as well.

The apparatus 1 includes batteries (not visible in the drawings), for example of the rechargeable type, and a charge port 9 is preferably included in body 2. The body 2 may also further include an input/output port, such as a USB port 8, for the exchange of data between the apparatus 1 and an external control unit, such as a computer (not depicted in the drawings).

The apparatus 1 may also include a standard on/off switch 6 and a display 7, which could be used as a touch screen as well, for visualizing information relating to the treatment. Apparatus 200 includes a touch screen or display 7 as well.

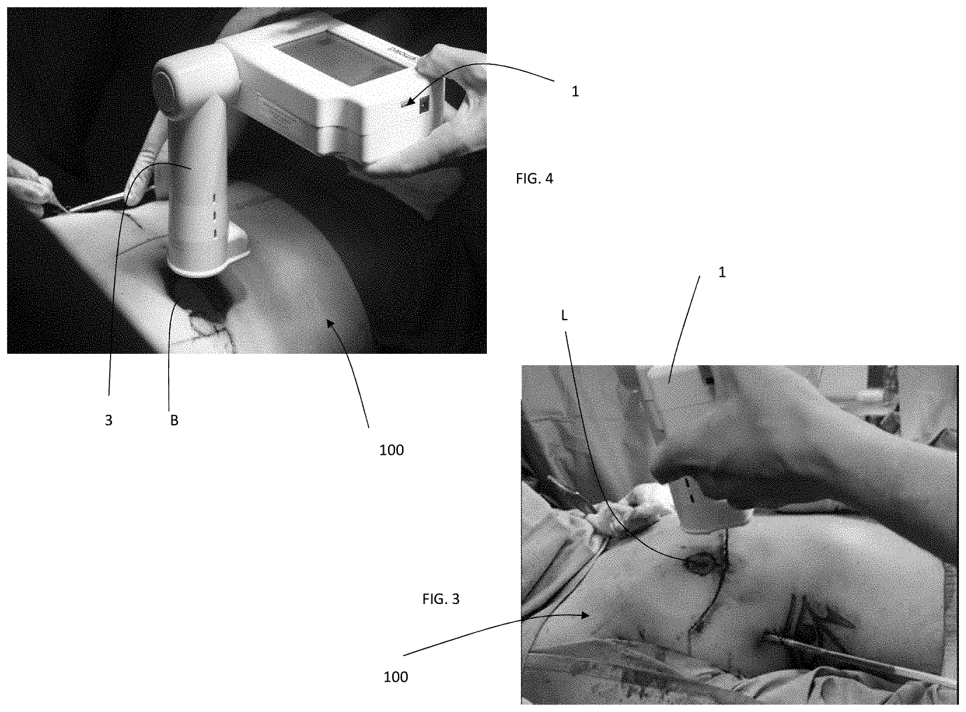

The apparatus 1, 200 is used for the treatment of a skin lesion L on a patient 100, as depicted in FIGS. 3 and 4. As shown in FIG. 3, first of all the localization of the lesion L on the patient 100 is made. The lesion defines an outer surface. The apparatus 1, 200 is thus suitably positioned so that the head 3 is substantially in front of the outer surface of the lesion L. An optimal distance between the head 3 where the LEDs are located and the lesion L is of about 3-6 cm.

On the basis of several characteristics of the lesion L itself and of the patient, the lesion L is irradiated by a beam B of electromagnetic radiation emitted by the apparatus 1, 200.

The treatment is repeated several times, for example twice a week, for a maximum of 10 times.

### Example 1

A 59 years old woman (in the following, “the patient”) suffered severe burns in her right arm (30%) and right leg (20%). Both lesions have been treated with autologous skin grafts. 10 days after the surgery (grafts), the right arm started a treatment according to the invention, while the leg is not treated.

The treatment of the arm is as follows.

The patient has been treated using the apparatus 1 of FIGS. 1 and 2 irradiating the lesion (the lesion, due to its size, has been divided in “sub-parts” and each sub-part received the same treatment) at a distance of 4 cm from it with a wavelength of about 420 nm and a fluence of 120 mW/cm² for 60 seconds. The treatment has been repeated 4 times, twice a week. The electromagnetic radiation is a pulsed electromagnetic radiation having a frequency of 400 kHz. The irradiation of the skin takes place as depicted in FIGS. 3 and 4.

The results and the comparison between the scars and keloids formed in the arm and the leg has been made after 6 months and 9 months from the accident. In order to compare the resulting scars/keloids, the scar score table of Yeong and al. has been used. The scale is as follows:

1. Burn scar surface

| | | | | | |
|---|---|---|---|---|---|
| −1 | 0 | 1 | 2 | 3 | 4 |
| smooth | normal | rough | rough | rough | rough |

2. Burn scar border height

| | | | | | |
|---|---|---|---|---|---|
| −1 | 0 | 1 | 2 | 3 | 4 |
| Depressed | normal | raised | raised | raised | raised |

3. Burn scar thickness

| | | | | | |
|---|---|---|---|---|---|
| −1 | 0 | 1 | 2 | 3 | 4 |
| Thinner | normal | thicker | thicker | thicker | thicker |

4. Color differences between scar and adjacent normal skin

| | | | | | |
|---|---|---|---|---|---|
| −1 | 0 | 1 | 2 | 3 | 4 |
| Hypopigmented | normal | hyper | hyper | hyper | hyper |

After 9 months the results are listed in TABLE 1 (using the above scale):

TABLE 1

| | Arm (treated with the | Leg (untreated with the |
|---|---|---|
| | method of the invention) | method of the invention) |
| Scar’s surface | 2 | 4 |
| Scar’s thickness | 2 | 4 |
| Color difference | 1 | 2 |
| Sum | 5 | 10 |

From the above results, it can be clearly seen that the treatment with the method of the invention greatly improved the scar appearance after healing of the lesion, compared with the untreated scar formed in a very similar lesion (caused by the same insult on the same patient).

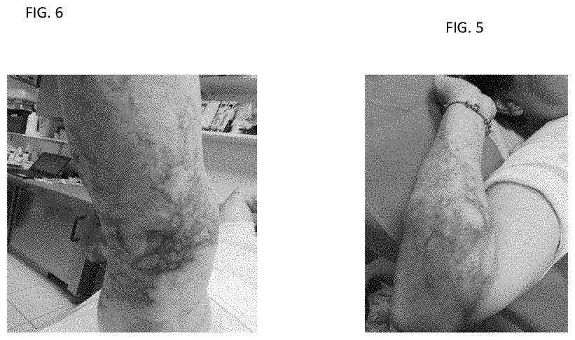

In FIGS. 5 and 6, the treated lesion (FIG. 5) using the method of the invention and the untreated lesion (FIG. 6) are shown.

### Example 2

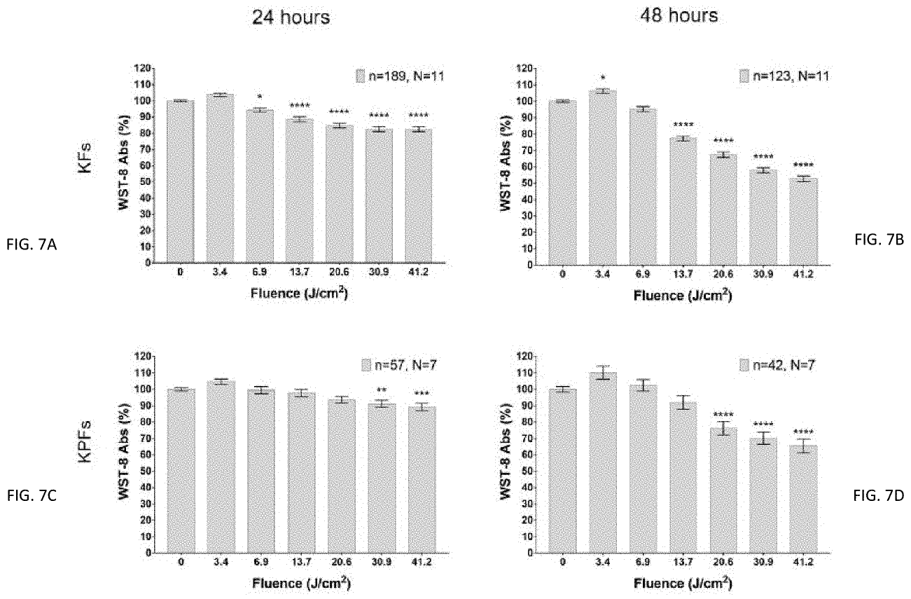

In order to understand why the present method is successful in preventing the formation of keloids and scars and minimizing the regrowth if already formed, the Applicant has performed the following in vitro tests on fibroblasts harvested on patients having different types of lesions. The following graphs of FIGS. 7-9 have been obtained according to the following methods.

Human fibroblasts, isolated from keloids and perilesional tissue, in vitro, have been irradiated with blue LED light.

The blue LED apparatus utilized in these experiments is based on commercially available LED, emitting at around 420 nm with 1 W (Watt) optical emission power. The apparatus 200 includes a bench top device furnished with a flexible polymeric fiber, 1.2 m in length, equipped with a touch screen 7 where it is possible to control all the irradiation parameters, such as irradiation time and power. The illuminated area corresponded to a circle of 5 mm in radius. The resulting power density was 1.2 W/cm². The intensity distribution of the light at the cells surface is homogeneous, as from a top hat intensity light source. The fluence values are calculated taking into account the dimension of the cells support and the irradiation spot, at the maximum energy provided by the device. The used electromagnetic radiation is a pulsed electromagnetic radiation at 400 KHz pulsed frequency.

All the irradiation parameters are measured using a photodiode energy sensor (Ophir, Darmstadt, Germany). The fluence values which correspond to different doses of blue light directly applied to cultured cells are reported in Table 2. These fluence values are measured at the fibroblast level, i.e. where the fibroblast are positioned.

TABLE 2

| Time (s) | Fluence (J/cm²) |
|---|---|
| 5 | 3.43 |
| 10 | 6.87 |
| 20 | 13.7 |
| 30 | 20.6 |
| 45 | 30.9 |
| 60 | 41.2 |

The fibroblast cells which are irradiated are coming from the tissue of several patients. All subjects provided written informed consent approved by the Hospital Ethical Board of AOU, Città della Salute e della Scienza, at the “Le Molinette” Hospital (Turin, Italy). All the experiments are performed in accordance with the Helsinki declaration in the matter of ethical principles and privacy of the human subjects. The differential diagnosis from hypertrophic scars and the score assigned in Fitzpatrick scale were assessed by the aesthetic surgeon. Eleven keloid samples were obtained from eleven patients (nine males and two females, average age 32.8±10.32 SD) subjected to reconstructive and aesthetic surgeries. From eight out of eleven patients, it was possible to obtain keloid perilesional tissue. In eight patients, the keloid tissue was already removed during previous manual surgeries (five patients) or laser-assisted surgeries (three patients). The general and clinical characteristics of all excised-keloid tissues are depicted in Table 3.

TABLE 3
Characteristics of keloid and perilesional tissue
used to prepare primary cultures of human fibroblasts.

| Fitzpatrick | | | |
|---|---|---|---|
| score | Year of Birth/Sex | Anatomical site/Size (cm) | Surgery |
| IV | 2004/m | hand/8 × 4 and perilesional tissue | surgical removal |
| II | 1972/m | helix/1.5*1 | Cryoexcision |
| II | 1975/f | earlobe/1*1 | Cryoexcision |
| II | 1987/m | gluteus/6*7 and perilesional tissue | surgical removal |
| II | 1987/m | scapula/5*5 and perilesional tissue | surgical removal |
| V | 1980/f | auricle/4*2 and perilesional tissue | Cryoexcision |
| V | 1980/m | helix/12*7 | Cryoexcision |
| VI | 1989/m | nape/2*3.5 and perilesional tissue | Cryoexcision |
| V | 1999/m | mandibular/5*1.5 and perilesional tissue | Cryoexcision |
| V | 1982/m | mandibular/2.5*2.5 and perilesional tissue | Cryoexcision |
| V | 1977/m | mandibular/8*3 and perilesional tissue | Cryoexcision |

Immediately after biopsy, both keloid and perilesional tissues were collected in Dulbecco Modified Eagle Medium (DMEM, Pan-React Applichem, Milan, Italy), maintained at 4° C. and used within 5 hours from the explant. Each sample was subjected at least to 6 vigorous washes to remove hair and blood residues. After that, keloids and perilesional tissues were dissected with scalpels or scissors to obtain sections of about five mm in diameter. The fragments were disposed on scratched-Petri dishes keeping semi-opened under laminar flow for about 40 minutes to allow the adhesion of each sample to the plate. After this procedure, low glucose DMEM supplemented with 10% Fetal Bovine Serum (FBS), 1% of Glutamine and 1% of Penicillin-Streptomycin (both purchased by EuroClone, Milan, Italy) and the cells were kept at 37° C. and 5% CO2. Fibroblasts migrated from the tissue to the bottom of the dish and within three weeks from the preparation of the cultures, fibroblasts were detached by Trypsin-EDTA 0.25% solution (Sigma-Aldrich, Milan, Italy), collected in a centrifuge tube, centrifuged at 1000 rpm for 6 minutes and the pellet was seeded in T25 flask (Greiner Bio-One, GmbH, Germany). The cells were maintained under standard culture conditions (37° C.; 5% CO2) and medium refreshed every 48 hours. Cells were splitted in T75 flask (Greiner Bio-One, GmbH, Germany) when reaching about 80% of confluence.

Cellular metabolic activity was measured by Cell Counting Kit-8 (CCK-8) assay (Sigma-Aldrich, Milan, Italy). The CCK-8 use WST-8, which produces a water-soluble formazan dye according to the dehydrogenase activity in the presence of 1-Methoxyphenazine methosulfate, a stable electron carrier mediator between NAD(P)H and tetrazolium dyes. Colorless WST-8 is bioreduced by cellular dehydrogenases and becomes WST-8 formazan with an orange color that is soluble in the tissue culture medium. CCK-8 assay was used by following the manufacturer's instructions. Fibroblasts cells were counted using Neubauer chamber and 5×103 were seeded in 96-multiwell plates (Greiner Bio-One, GmbH, Germany) and maintained for 24 hours in standard culture conditions (37° C. and 5% CO2) before the experiments. To avoid interference, such as double or partial irradiation, cells were seeded in alternate wells, in rows and columns, for each multiwell. The day after the treatment, DMEM was replaced with SFM DMEM and the cells were irradiated by the blue LED light applying the followed doses in 18 wells: 3.43-6.87-13.7-20.6-30.9 and 41.2 J/cm². For each experiment, three wells were left untreated and used as a control, other three wells were used to measure the background of multiwell. The irradiation was performed by holding the apparatus 200 steadily 1 cm far from the bottom of each well. The 1 cm is calculated from the tip of the optical fiber 15 and the bottom of the well. The absorbance at 450 nm was read using a reference wavelength at 630 nm and was evaluated using an automatic microplate absorbance reader (LT-4000 Labtech, Heathfield, East Sussex, England). Each experiment was performed at least in duplicate.

This protocol enabled to study the dose of irradiation that can provide the best modulation of fibroblasts, if any. For each experiment, three wells were left untreated and used as a control.

The treatment with the blue LED light induces a significant decrease in keloid fibroblasts metabolism, measured with WST-8 (tetrazolium salt) reagent above described. This effect is dose-dependent, starts 24 hours from the irradiation (FIG. 7A), and becomes more pronounced after 48 hours (FIG. 7B). In particular, at 24 hours after the treatment, the decrease reaches significant levels at fluence values ranging from 6.87 to 41.2 J/cm² (FIG. 7A); while at 48 hours, a significant decrease is reached at fluences from 13.6 to 41.2 J/cm² (FIG. 7B). Noteworthy, the lowest dose tested (3.43 J/cm²) causes a significant increase in fibroblast metabolism 48 hours after irradiation (FIG. 7B). A significant decrease in cell metabolism is observed also in fibroblasts isolated from keloid perilesional tissues, both 24 and 48 hours after blue LED light irradiation (FIGS. 7C, 7D). In detail, FIG. 7C shows a significant reduction in cell metabolism at fluences in the range from 30.9 to 41.2 J/cm², while after 48 hours, even lower doses are effective in reducing cell metabolism (in the range 20.6-41.2 J/cm²; FIG. 7D).

Cell proliferation was evaluated by Sulforhodamine B based (SRB) assay purchased from Sigma-Aldrich (Milan, Italy). Sulforhodamine binds stoichiometrically to proteins under mildly acidic conditions and then can be extracted under basic conditions; thus, the amount of bound dye can be used as an approximation of cell mass, which can then be extrapolated to measure cell proliferation. The human keloid fibroblast cells were counted, maintained and treated with the blue LED light using the same protocol as CCK-8, described above. The absorbance at 570 nm was read using a reference wavelength at 630 nm and was evaluated using an automatic microplate absorbance reader (LT-4000 Labtech, Heathfield, East Sussex, England). Also, the experiments were performed at least in duplicate.

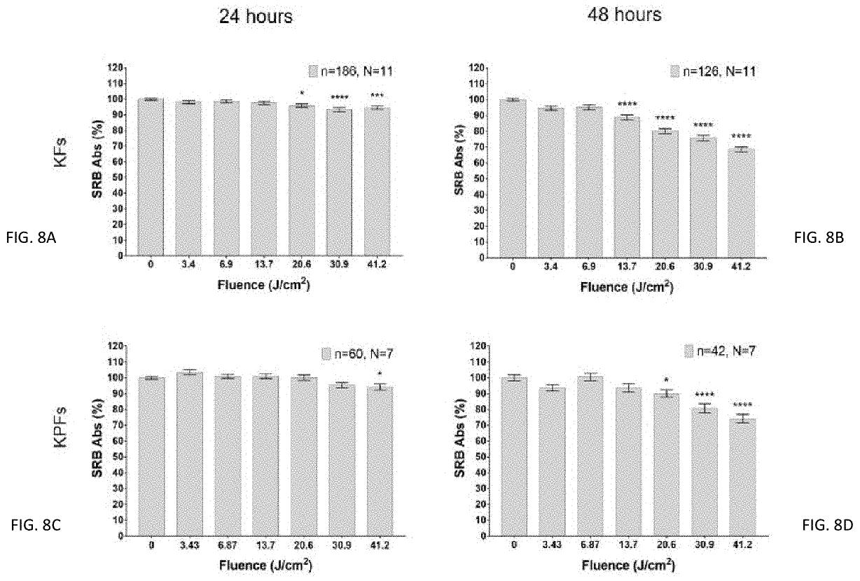

Cultured fibroblasts isolated from keloid tissues reduce their proliferation rate with an application of the blue LED light fluence in the range from 20.6 to 41.2 J/cm² (FIG. 8A), as observed 24 hours after treatment. This effect is even more pronounced 48 hours after the treatment, when the Sulforhodamine B (SRB) absorbance decreases in correspondence to fluence values ranging from 13.9 to 41.2 J/cm² (FIG. 8B). The reduction of fibroblasts proliferation modulated by the application of the blue LED light occurs in a strictly dose-dependent manner. In fibroblasts isolated from keloid perilesional tissues, only the higher dose of irradiation with blue LED light induces a decrease in SRB absorbance after 24 hours (FIG. 8C), while at 48 hours, the doses of 20.6, 30.9 and 41.2 J/cm² can significantly reduce SRB absorbance (FIG. 8D).

To confirm the results obtained with the tests carried out on metabolism and proliferation, a cell viability assay by testing the highest dose of blue LED light has been performed. Cell viability was assessed by using Trypan Blue solution 0.4% (Sigma-Aldrich, Milan, Italy) which is not absorbed by healthy and viable cells, but that stains cells with a damaged membrane, in blue or light blue color. Fibroblast cells were seeded in 35 mm² dishes (Greiner Bio-One, GmbH, Germany) and were maintained in low glucose DMEM for 24 hours. Treated samples were irradiated with the blue LED light applying 41.2 J/cm², whereas control samples were kept not irradiated. To perform these experiments, we use an appropriate source of blue LED light, capable to cover the entire plate area. The treatment was performed in serum-free medium (SFM DMEM) without red phenol, to avoid possible interference during the irradiation. All the dishes were divided into 2 groups and were evaluated 24 or 48 hours after irradiation; during this time cells were maintained under standard culture conditions (37° C. and 5% CO2). After the removal of the SFM DMEM, 2 washes in PBS were performed and Trypan Blue solution diluted 1:4 in PBS was applied for 6 minutes to all the dishes. After 2 additional washes in PBS, cells were immediately observed under an inverted optical microscope (Eurotek Orma, INV100T, Milan, Italy) using 10× magnification. Ten random images for each sample were acquired using a 5-megapixel photo-camera (Eurotek Orma, Milan, Italy) and Manta software (MANTA, New York, USA) was used to analyze the collected images. Two separate and blind counts were carried out by trained personnel. The experiment was performed at least in triplicate.

The dye exclusion test was used to determine the number of total viable cells counted in the treated and in the control samples after 24 and 48 hours. After the staining, no stained cells in any sample has been found. However, the number of cells in some samples was visibly lower than in others. For this reason, 10 random images were captured from each sample and cells were counted. Data obtained were then compared between controls and treated and can be viewed in Table 4. The results demonstrated that the application of 41.2 J/cm² significantly reduces the number of total keloid fibroblasts, 48 hours after the treatment with the blue LED light, while at 24 hours from the treatment, no significant difference are found in respect to untreated samples. The same experimental procedures were also performed in fibroblasts isolated from perilesional tissue. In this case, our results showed that after 24 hours, a 41.2 J/cm² fluence induces a decrease in the total number of fibroblasts cell, in comparison to control sample. This result demonstrates that the reduction observed by applying the fluence of 41.2 J/cm² in WST-8 and SRB assays, should be ascribed to a cytotoxic effect.

TABLE 4
Pooled data of cell viability in keloid and perilesional fibroblasts (mean ± SD) in
control and treated cell cultures, and the relative p values resulting from the unpaired t-test.

| Sample | Control | | Treated | J/cm² | p value |
|---|---|---|---|---|---|
| Keloid 24 h | 36.55 | (17.91) | 37.37 | (10.42) | 0.761 |
| Keloid 48 h | 55.83 | (17.39) | 40.40 | (15.56) | <0.001 |
| Perilesional 24 h | 53.20 | (17.94) | 29.62 | (11.02) | <0.001 |
| Perilesional 48 h | 53.38 | (17.57) | 24.13 | (12.91) | <0.001 |

Data obtained from CCK-8, SRB assays were expressed as mean±SEM (Standard Error of the Mean). Student's paired or unpaired t-tests and one-way ANOVA 360 followed by Dunnett's multiple comparison test analysis were performed. All data were analyzed by using commercial software package GraphPad Prism (GraphPad Software, San Diego, Calif., USA). The analysis of cells viability experiments was performed as follows: the hypothesis of normality was verified using the Kolmogorov-Smirnov test. This allowed us to evaluate the most suitable type of statistical analysis. This led to the choice of the two-sample two-tailed t-test to verify the equality of the treated/control means separately at 24 and 48 hours. A variance analysis of repeated measures was then performed for a multiple comparison between 24 and 48 hours. Through the Bartlett test it was found that the assumption of homoscedasticity in the variables is not respected and this led us to choose a non-parametric approach by opting for the Friedman test. This analysis was performed using Rcommander open-source software (https://www.rcommander.com/). Statistical significance was set at *p<0.05 for all the experimental results.

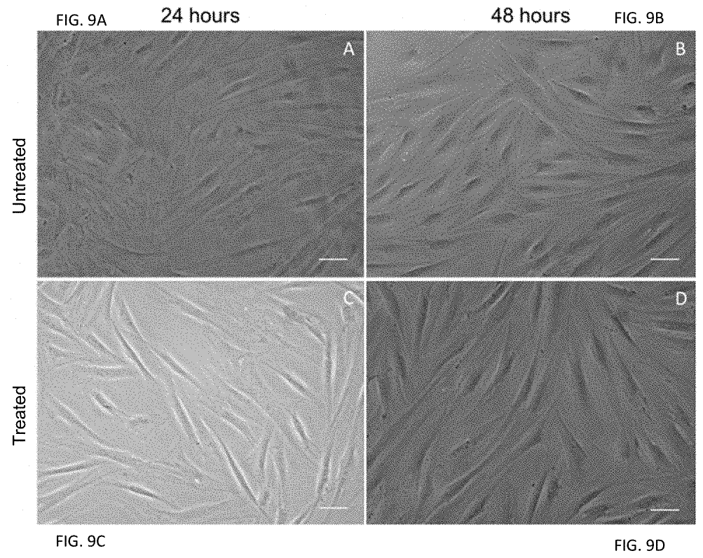

From the above, it is clear that blue light can modulate both cell metabolism and proliferation in a dose-dependent manner and that a fluence of 41.2 J/cm² or above induces a reduction in cell viability, with different timings in keloid-derived fibroblasts and in fibroblasts isolated from perilesional tissues. The results demonstrate that after only 24 hours from the irradiation, the blue LED light can modulate both cell metabolism and proliferation in a dose-dependent manner in either type of fibroblasts; after 48 hours this effect is still ongoing and appears even more accentuated. A trypan blue exclusion test on fibroblast cultures after 24 and 48 hours from the application of the maximum dose (41.2 J/cm²) has been performed. The results (see FIGS. 9A-D) did not show colored cells, a clue of cellular death, both in fibroblasts isolated from keloids and from perilesional tissues, at either time tested. Aimed to explore this evidence, we evaluated the number of cells before and after the treatment with blue LED light. It has emerged that the number of cells in controls and treated samples did not change after 24 hours after the irradiation in keloid-derived fibroblasts, while at 48 hours, the number of treated cells was significantly reduced in comparison to untreated samples. In fibroblasts isolated from perilesional tissues the number of treated cells was significantly decreased by almost 50% already 24 hours after the treatment. This result demonstrates that the reduction observed by applying the fluence of 41.2 J/cm² in WST-8 and SRB assays, should be ascribed to cell death.

The same tests above described have been performed using fibroblasts harvested from healthy human skin. The healthy human skin samples were obtained from 7 healthy patients subjected to mole removal. Surgeries were performed at the Azienda Ospedaliera Università degli Studi di Perugia (Italy). The study was approved by the Hospital Ethical Board (16806/19/AV, Jul. 17, 2019). All the experiments were performed in accordance with the Helsinki declaration and in conformity with Good Clinical Practice (GPC). After the biopsy, healthy skin tissues were immediately frozen at −80° C. in DMEM. To prepare primary cultures, the samples were thawed at 37° C. and fragmented into small pieces. Each specimen was collected in a scratched-plated (Greiner Bio-One, GmbH) and kept under laminar flow carefully avoiding the dehydration until the adhesion to the plate occurred [21]. After this procedure, DMEM low glucose (1.5 g/L) medium (Pan-React Applichem, Milan, Italy), supplemented with 10% FBS, 1% of Glutamine and 1% pen/strep (EuroClone, Milan, Italy), was added and cells maintained at (37° C. and 5% CO2). Within three weeks from the preparation, fibroblasts migrated out of the tissue. When fibroblasts reached confluence, the cells were detached using Trypsin-EDTA solution (Sigma-Aldrich, Milan, Italy), collected in a centrifuge tube, centrifuged and the pellet was seeded in T25 flask (Greiner Bio-One, GmbH). Fibroblasts were maintained under standard culture conditions ((37° C. and 5% CO2)) in T75 flask (Greiner Bio-One, GmbH) and the medium was refreshed every 48 hours. Cells were split when reaching about 80% of confluence.

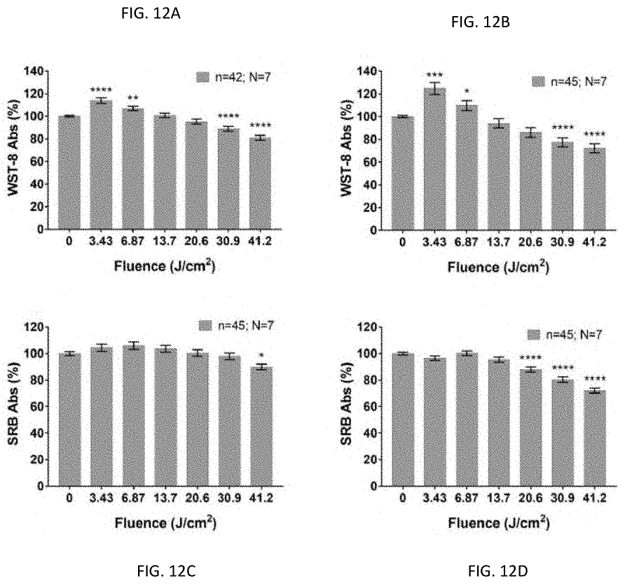

The irradiated fibroblasts show a dual response to blue light. Indeed, low doses (3.43 and 6.87 57 J/cm²) stimulate an increase in metabolic activity, while higher doses (30.9 and 41.2 J/cm²) reduce cell metabolism (FIG. 12A). The same effect is more pronounced 48 hours after treatment (FIG. 12B). Proliferation shows a reduction only at the highest dose, 24 hours from the application, while doses ranging from 20.6 to 41.2 J/cm² can affect fibroblasts proliferation 48 hours after the irradiation (FIG. 12C-12D).

The SRB and WST-8 essays are obtained as above for the keloid and perilesional fibroblasts.

Trypan blue staining was performed both in fibroblasts and in keratinocytes after the application of 41.2 J/cm² fluence dose of blue LED light. In Table 5 are reported values of cell viability before and after irradiation after the hypothesis of normality was verified using the Kolmogorov-Smirnov test. We observed a significant reduction of cell viability in the fibroblasts cultures 24 hours after the treatment, while at 48 hours, no significant differences were found in respect to untreated samples.

TABLE 5
Pooled data of cell viability in healthy fibroblast cells at 24 and 48 hours after the application
of 41.2 J/cm². Data are expressed as mean and SD (in brackets), two-sample two-tailed t-test

| Sample | Control | | Treated | | p value |
|---|---|---|---|---|---|
| Fibroblast 24 h | 93.20 | (20.05) | 44.38 | (21.84) | <0.001 |
| Fibroblast 48 h | 39.52 | (17.08) | 38.65 | (16.51) | 0.778 |

### Example 3

From the experiments above, it is clear that the blue light at the claimed 410-430 nm wavelength alters the behavior of fibroblasts in scars and keloids. In order to obtain the correct fluence to be applied to the human skin (the above experiments have been performed in vitro), experiments have been performed where the fibroblasts described with reference to the Experiment 2 have been covered by a layer of synthetic skin. The synthetic skin which has been used is Integra® Dermal Regeneration Template as described in the web site ww.integra.com.

Integra® Dermal Regeneration Template (Integra Template) has two layers: a thin outer layer of silicone and a thick inner matrix layer of pure bovine collagen and glycosaminoglycan (GAG). Both collagen and GAG are normal components of human skin. In Integra, the collagen is obtained from bovine tendon collagen and the glycosaminoglycan is obtained from shark cartilage. The silicon layer has been removed with a surgical tool, so that a layer “similar” to the dermis is left. The thickness of this layer (i.e. the layer used in this experiment) is of about 0.5 mm.

Below this layer of “dermis”, the same cells as the healthy cells described at the end of Example 2 (the results of which are depicted in FIGS. 12A-D) have been positioned. Therefore, the same fibroblasts from the same donors as the healthy cells described at the end of Example 2 are hereby considered.

The apparatus 200 including an optical fiber was used to irradiate the samples, setting 100% of the emitted power (that is, setting a power of 150 mw/cm²). The electromagnetic radiation is pulsed at 400 kHz. The apparatus 200 and its settings are identical to those of Example 2. The exposure times (=treatment times) tested are: 30 s, 1 min, 2 min, 4 min, 8 min. The experiment was conducted in duplicate. The power emitted by the apparatus 200 was measured by placing the fiber at a distance from a distance sensor (powermeter Ophir) equal to the distance of the multiwell plane (on which the fibroblasts adhere, see example 2) from the tip of the fiber emitting the electromagnetic radiation at 420 nm±10 nm. About 150 mW of power arrive at the bottom of the multiwall when only the fibroblasts are present (i.e. they are not covered by anything), on a spot of about 6 mm in diameter. The measurement was repeated in order to verify how much power passes through the thickness of synthetic skin (without silicone layer): it has been found that this skin substitute filters about 50% of the incident power, so that about 75 mW of optical power reaches the fibroblasts at the bottom of the multiwall when covered by the Integra “dermis”. The skin substitute has no other type of cells and obviously is not supplied with blood vessels, so it is more transparent than an identical thickness of real donor dermis, although this is a good approximation.

Two series of measurements were made: the first by irradiating the cell cultures directly, the second by irradiating the multiwells in which the fibroblasts are located, covered by a portion of synthetic skin. In this way, irradiation is simulated in physiological conditions of intact skin. Note that under real conditions, fibroblasts are found at various depths in the dermis. Using the dermal substitute scheme placed over a cell layer, the case in which the fibroblasts are at a great depth (500 microns deep from the surface layer) is simulated or in those sites where the skin is thicker. The closer the fibroblasts are to the outer skin wall (i.e. towards the epidermis), the greater the power of light to which they are exposed is.

The cells irradiated in the manner described above, immediately after the treatment, were stored in an incubator for 24 h and then their viability was studied.

The cells were observed under an optical microscope for a qualitative analysis of morphology.

From the observations under the microscope, the fibroblasts, for an exposure time longer than 2 minutes, seem to lose their characteristic elongated shape, appear dark in color and with cytoplasmic granules. The effect is very marked in the sample irradiated for 8 minutes. It therefore appears that irradiation times longer than 2 minutes (direct irradiation) may cause damage to fibroblasts.

The same samples were then subjected to analysis with WST-8, in the same way as described in Example 2, in order to have a measure of the metabolism of the fibroblast cells after irradiation.

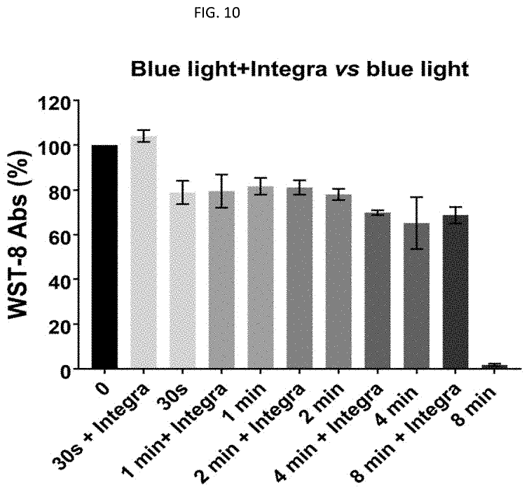

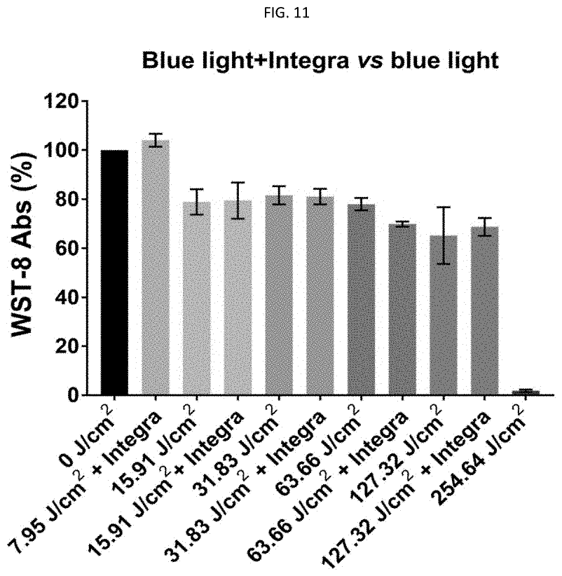

The results of the two experiments (in both fibroblasts covered by synthetic skin, Integra, and direct irradiation, that is, fibroblasts as in Example 2) with varying irradiation time are shown in FIGS. 10 and 11.

The results of FIG. 10 are the results of the irradiations for different exposure times, with or without the synthetic layer above the fibroblasts. The results of FIG. 11 are the same as those of FIG. 10 where, instead of the exposure times, the fluences at the fibroblast level is measured. As shown, the fluence in case of the synthetic skin that reaches the fibroblasts is substantially 50% of the fluence without the synthetic skin.

The abscissa in FIGS. 10 and 11 is as follows. When in addition to the units (either seconds, minutes or J/cm²) a “+ integra” is mentioned, it means that the measurement is made in a sample which also comprises the Integra synthetic skin covering the fibroblasts. If nothing is mentioned (just the units are written), then the measurement refers to a sample containing the fibroblasts without the synthetic skin.

TABLE 6

| | | Mean (SD) blue | | Mean (SD) | | |
|---|---|---|---|---|---|---|
| Fluence | | light + integra | | blue light | | p-value |
| 15.91 | J/cm² | 79.44 | (10.39) | 78.89 | (7.33) | 0.957 |
| 31.83 | J/cm² | 81.10 | (4.51) | 81.62 | (5.24) | 0.925 |
| 63.66 | J/cm² | 69.89 | (1.47) | 78.01 | (3.52) | 0.095 |
| 127.32 | J/cm² | 68.75 | (5.18) | 65.22 | (16.34) | 0.798 |

The table 6 above summarizes FIGS. 10 and 11.

Based on the analysis carried out so far, and by a comparison of the behavior of healthy fibroblasts covered or not by the synthetic skin (FIG. 12A should be compared with FIG. 11) it can be said that: the modulating effect of cellular activity in fibroblasts from healthy human donors is also observable when these fibroblasts are located under a layer of tissue such as the skin substitute Integra® Dermal Regeneration Template. The modulating effect of cellular activity is similar to that observed in fibroblasts from a healthy human donor directly irradiated, with the same dose of light on the cells. To send the same dose of light to fibroblasts that are deep in the skin, the irradiation times must be increased (compared to direct irradiation of cultured fibroblasts).

It is understood, therefore, that this invention is not limited to the particular embodiments disclosed, but is intended to cover all modifications which are within the spirit and scope of the invention as defined by the appended claims; the above description; and/or shown in the attached drawings.

1. A method to prevent formation of a scar or keloid following an insult to an area of a mammalian skin creating a lesion defining an outer surface, wherein the lesion reaches at least the dermis of the skin, the method comprising:
directly irradiating the lesion with electromagnetic radiation having a wavelength comprised between 410 nm and 430 nm for an exposure time long enough that an energy density received by the outer surface of the lesion is comprised between 15 J/cm² and 120 J/cm².

2. A method of treatment of an already formed scar or keloid on mammalian skin to avoid its recurrence, the method comprising:
surgically removing the scar or keloid to form an area of the skin with a lesion defining an outer surface which reaches the dermis; and
directly irradiating the lesion with electromagnetic radiation having a wavelength comprised between 410 nm and 430 nm for an exposure time long enough that the energy density received by the outer surface of the lesion is comprised between 15 J/cm² and 120 J/cm².

3. The method according to claim 1, further comprising:
repeating the irradiation of the lesion between 3 times and 10 times.

4. The method according to claim 3, wherein between an irradiation and the subsequent irradiation, the lesion is not irradiated for at least 48 hours.

5. The method according to claim 3, further comprising:
providing a recover time interval comprised between 48 hours and 96 hours between irradiations.

6. The method according to claim 1
wherein the outer surface of the lesion is irradiated for an exposure time comprised between 30 seconds and 600 seconds.

7. The method according to claim 1
wherein the outer surface of the lesion is irradiated so that the radiant flux received by the outer surface per unit area of the lesion is comprised between 50 mW/cm² and 200 mW/cm².

8. The method according to claim 1, wherein the step of irradiating the wound includes:
irradiating the outer surface of the lesion with electromagnetic radiation emitted by one or more LEDs.

9. The method according to claim 8, wherein the distance between the one or more LEDs and the outer surface of the lesion is comprised between 1 cm and 15 cm.

10. The method according to claim 1, wherein the step of irradiating the lesion is performed in such a way that the radiant flux received by the outer surface of the lesion per unit area (W/m²) remains constant during the whole irradiation.

11. The method according to claim 1, wherein the mammalian skin is human skin.

12. The method according to claim 1, wherein the lesion is caused by surgery.

13. The method according to claim 1, wherein the step of irradiating the lesion with electromagnetic radiation includes irradiating the lesion with a pulsed electromagnetic radiation.

14. The method according to claim 13, wherein the pulsed electromagnetic radiation has a repetition rate comprised between 10 kHz and 400 KHz.

15. The method according to claim 2, further comprising:
repeating the irradiation of the lesion between 3 times and 10 times.

16. The method according to claim 15, wherein between an irradiation and the subsequent irradiation, the lesion is not irradiated for at least 48 hours.

17. The method according to claim 15, further comprising:
providing a recovery time interval comprised between 48 hours and 96 hours between irradiations.

18. The method according to claim 2
wherein the outer surface of the lesion is irradiated for an exposure time comprised between 30 seconds and 600 seconds.

19. The method according to claim 2
wherein the outer surface of the lesion is irradiated so that the radiant flux received by the outer surface per unit area of the lesion is comprised between 50 mW/cm² and 200 mW/cm².

20. The method according to claim 2, wherein the step of irradiating the wound includes:
irradiating the outer surface of the lesion with electromagnetic radiation emitted by one or more LEDs.
